(19) United States
(12) Patent Application Publication
(10) Pub. No.: US 2025/0090857 A1
(43) Pub. Date: Mar. 20, 2025

(54) ULTRAVIOLET B LIGHTING SYSTEM AND METHOD

(71) Applicant: KORRUS, INC., LOS ANGELES, CA (US)

(72) Inventors: BENJAMIN HARRISON, LOS ANGELES, CA (US); PAUL KENNETH PICKARD, LOS ANGELES, CA (US)

(21) Appl. No.: 18/971,375

(22) Filed: Dec. 6, 2024

Related U.S. Application Data
(63) Continuation of application No. PCT/US23/25116, filed on Jun. 13, 2023.
(60) Provisional application No. 63/352,099, filed on Jun. 14, 2022.

(51) Int. Cl. A61N 5/06

(52) U.S. Cl. CPC A61N 5/0616; A61N 5/0618; A61N 2005/0626; A61N 2005/064; A61N 2005/0659; A61N 2005/0661; A61N 2005/0663

(57) ABSTRACT

A lighting system for emitting emitted light, said light system comprising: (a) a first light source for emitting a first light; (b) a second light source for emitting UVB light, wherein said emitted light comprises a combination of said first light and said UVB light; and (c) a controller for said second light source configured to deliver a dose of said UVB to at least one target sufficient to cause vitamin D synthesis in said target.

### REFERENCE TO RELATED APPLICATION

This application is based on U.S. Provisional Application No. 63/352,099, entitled ULTRAVIOLET B LIGHTING SYSTEM AND METHOD, filed Jun. 14, 2022, and is incorporated herein by reference.

### FIELD OF INVENTION

This invention relates, generally, to light therapy, and, more specifically, to a method and system for ultraviolet B (UVB) light therapy.

### BACKGROUND

The use of light therapy for the treatment of a host of medical conditions has been widely studied, with new use cases examined and outcomes documented in literature. Of particular interest herein is ultraviolet B (UVB) light therapy on people to release healthy photoproducts for the treatment of vitamin D deficiency, seasonal depression and psoriasis, among others.

By way of background, there are three UV light types, and each has a different impact on the human body. First, UVA light (315 nm to 400 nm) accounts for the majority of UV radiation that reaches the earth's surface. Approximately 95% of the sun's rays reaching the earth's surface are UVA. UVA wavelengths trigger melanin production which darkens the skin. But UVA is also associated with premature aging, wrinkles and skin cancer, as it penetrates deep into the layers of the skin. Second, UVB light (280 nm to 315 nm) is mostly absorbed by the ozone layer. Roughly 5% of UVB rays that reach the earth's surface, and the intensity at which exposure occurs is dependent on season, geography, and time of day, among other factors. UVB light therapy has long been used to treat a variety of medical conditions, such as chronic skin conditions and vitamin D deficiency. UVC light (100 nm to 280 nm) is the most harmful type of UV light. Exposure to UVC light is mostly avoidable, as this spectrum is almost completely absorbed by Earth's atmosphere.

When human skin is exposed to the sun's rays, hormones and peptides are naturally activated within the body that contribute to systemic health and wellness. Additionally, sun exposure produces mood-boosting beta endorphins responsible for feelings of happiness and contentment. These substances made from chemical reactions with sunlight are called photoproducts.

These healthy photoproducts stimulated by UVB light can be used to treat a variety health problems. For example, UVB is known to treat vitamin D deficiency. Vitamin D3 is a hormone naturally produced by sun to skin contact. Levels of vitamin D are managed by several internal control mechanisms to ensure the body gets only what it needs. A UVB exposure deficiency results in the body failing to absorb the necessary light to produce vitamin D, which supports immune system health. Deficiency in vitamin D leads to weakened immune responses. UVB can also be used to treat seasonal depression, which is a mental condition that occurs when there is less sunlight at certain times of the year. UVB is also an effective treatment for Psoriasis, which is a chronic skin disease that manifests as red scaly plaques commonly found on the elbows, knees, and scalp. UVB penetrates the skin and slows the growth of affected skin cells that are causing the scaly patches.

Because of the uncontrollable variables impacting how much UVB a person receives (e.g., atmospheric conditions, geographic location, season of the year, time of day, time spent outside, etc.), and because many people tend to use sunscreen to protect against overexposure to UV rays, UVB light therapy provides people with a safe and effective way to get the UV exposure their bodies need. Such therapy is often provided in a medical setting. UVB light therapy may use fluorescent bulb technology or LED light technology. Ongoing LED innovation has enabled delivery of narrow spectrum UVB light treatments for optimal results. Still other innovations are needed to optimize the benefits of UVB therapy. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Although exposure to ultraviolet B (UVB) is known to cause the synthesis of vitamin D in humans and other benefits as described above, Applicant recognizes that UVB combined with a second light source can synergistically improve the effectiveness of UVB light therapy in releasing healthy photoproducts, and improve a person's health generally.

For example, in one embodiment, UVB is combined with long red or near infrared (NIR) light to optimize the synthesis of vitamin D. More specifically, in this embodiment, Applicant recognizes that long red or NIR light tends to promote activation of enzymes, which, in turn, repair DNA damage that might be caused by UVB. Thus, while the UVB is promoting synthesis of vitamin D, the long red/NIR light is promoting healing of the DNA damage that might be caused by the UVB light. In one embodiment, the long red/NIR light is used to saturate the enzymes of the user prior to the user receiving the UVB dose. In one embodiment, the user receives the long red/NIR light in a private booth, thereby allowing the user to reveal more skin area, thus reducing the time needed to absorb red light or the UVB light dose.

In another embodiment, UVB light is combined with light therapy to treat Seasonal Affective Disorder (SAD). Applicant recognizes that a contributing factor to SAD might be low vitamin D. That is, there is evidence that during seasons of low light, very little vitamin D is synthesized, and people may be surviving off of vitamin D reserves. By providing UVB to promote vitamin D synthesis in addition to therapy light to counter SAD, the lighting system of the present invention provides a dual prong treatment of SAD.

In another embodiment, the UVB light is timed to minimize the harmful effects of UVB the light. More specifically, the harmful effects of UVB light appear to be minimized at biological midday. Accordingly, in one embodiment, the UVB light is delivered during biological mid-day, e.g. 5-9 hours after habitual wake time, or based on feedback from the user. In one embodiment, the lighting system of the present invention is configured to reinforce/realign a person's circadian rhythm before delivering the UVB dose.

Still other benefits of using UVB in combination with other light will be obvious to those in light of this disclosure.

### BRIEF DESCRIPTION OF FIGURES

FIG. 1 shows one embodiment of a lighting system of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

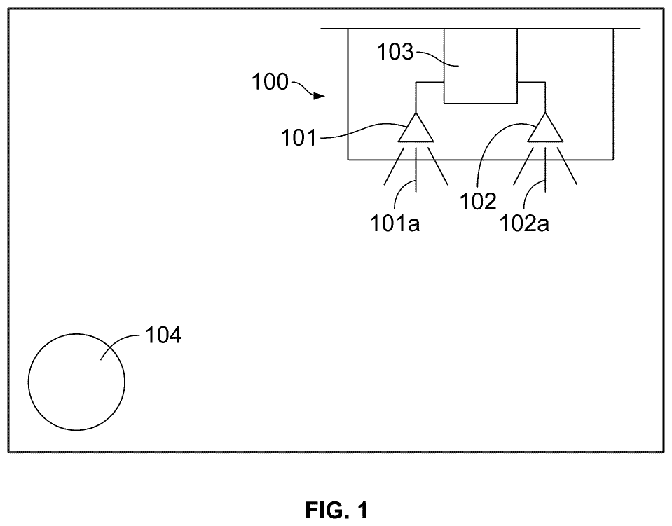

Referring to FIG. 1, one embodiment of a lighting system 100 of the present invention is shown. The light system 100 emits emitted light, and comprises: (a) a first light 101 source for emitting UVB light 101a; (b) a second light source 102 for emitting a second light 102a, wherein said emitted light comprises a combination of said second light and said UVB light; and (c) a controller 103 for controlling at least said first light source configured to deliver a dose of said UVB to at least one target 104 sufficient to cause the release of photoproducts in said target.

### First Light Source

The first light source functions to provide a therapeutic dose of UVB to a target in combination with the second light source. As used herein, the term “target” refers to any plant or animal capable of releasing photoproducts when exposed to UVB. The first light source may be any known UVB light source, although a light emitting diode (LED) light source may be preferred in certain embodiments. In one embodiment, the UVB light has a peak wavelength of 290-315 nm, and, in a more particular embodiment, the UVB light has a peak wavelength of about 300 nm. The UVB light source should be sufficiently powerful to deliver at least a 0.35 standard erythemal dose (SED) to said target.

By way of background, the SED is defined as 100 J m⁻². This is the integral of total UV energy per square meter multiplied by the CIE erythema reference action spectrum. The CIE erythema reference action spectrum is well known. Total energy per area may be calculated by multiplying average power by the duration of exposure. Similarly, average power can be calculated by dividing total energy by duration. For example, the power of an 0.35 SED over 8 hours can be determined as follows:

0.35 SED=35 J m⁻², and
over eight hours, this gives an average power of

$$\frac{35 {Jm}^{- 2}}{8 hours} = \frac{35 {Jm}^{- 2}}{28800 s} = 1.2 mW {m}^{- 2} .$$

Since the delivery of this UV may be coupled with visible light, the UV power can be specified in relation to the number of lumens produces by the first light. One way to do this is by relating UV irradiance to illuminance. Take, for example, a space lit to 500 lux. 500 lux is 500 lumens per square meter:

$$\frac{1.2 mW {m}^{- 2}}{500 lumen {m}^{- 2}} = 2.4 μ W / lumen .$$

Using this, one can determine that a 1000 lumen downlight should also emit 2.4 mW of CIE erythema reference action spectrum weighted UV-B.

### Second Light

The second light source functions to provide a second light, which, in combination with the UVB light, provides synergistic benefits. Again, Applicant recognizes that UVB combined with a second light source can synergistically improve the effectiveness of UVB light therapy (e.g., for vitamin D synthesis), and improve a person's health generally. In this disclosure, various embodiments of the second light are disclosed. It should be understood, however, this list is not intended to be exclusive but rather to illustrate how certain light combinations with UVB can provide for synergistic health benefits.

### Long Red or NIR Light

As mentioned above, Applicant recognizes that UVB combined with a second light source can synergistically improve the effectiveness of UVB light therapy (e.g., for vitamin D synthesis), and improve a person's health generally. In one embodiment, UVB is combined with long red or nearly infrared (NIR) light to optimize the synthesis of vitamin D. More specifically, in this embodiment, Applicant recognizes that long red or NIR light tends to promote activation of photoproducts (e.g. enzymes), which, in turn, repair DNA damage that might be caused by UVB. Without being tied to a particular theory, Applicant believes that long red or NIR light tends to activate cytochrome c oxidase enzymes. Thus, while the UVB is promoting synthesis of vitamin D, the long red/NIR light is promoting healing of the DNA damage that might be caused by the UVB light. Accordingly, in one embodiment, the second light comprising long red or NIR at a sufficient intensity at said target to be absorbed by cytochrome c oxidase enzymes.

In one embodiment, the controller is configured to emit said second light before the dose of UVB is delivered to said target, thereby building the target's defensed to the UVB prior to exposure to UVB. In one embodiment, the second light is administered before and after the UVB light is delivered. Such an embodiment mimics natural light exposure. More specifically, the protective and healing effects of NIR are relatively long timescale effects. Due to the transmission spectrum of the atmosphere and the variation in optical pathlength of sunlight traversing the atmosphere through the day we receive almost no UV in the earlier and later parts of the day, whereas we still receive lots of NIR at these times. The argument is that morning NIR prepares us for oxidative stress then the afternoon dose helps us to heal. In another embodiment, the long red/NIR light is used to saturate the enzymes of the user prior to the user receiving the UVB dose to maximize the target's resistance to the harmful effects of UVB before exposure.

In one embodiment, the user receives the long red/NIR light in a private booth, thereby allowing the user to expose more skin area to the long red/NIR light, thus reducing the time needed to absorb red light or the UVB light dose.

### UVB+SAD Therapy

In another embodiment, UVB light is combined with therapy light to treat Seasonal Affective Disorder (SAD). Applicant recognizes that a contributing factor to SAD might be low vitamin D. That is, there is evidence that during seasons of low light, very little vitamin D is synthesized, and people may be surviving off of vitamin D reserves. By providing UVB to promote vitamin D synthesis in addition to therapy light to counter SAD, the lighting system the present invention provides a dual prong treatment of SAD. Accordingly, in one embodiment, the second light is configured to alter said target's circadian rhythm to treat SAD. In one embodiment, said second light source has at least two modes, a first mode in which said second light is a high EML light to stimulate circadian response, and a second mode in which said second light is a low EML light to minimize a circadian response. In one embodiment, said dose of UVB is delivered when said second light source is in said first mode.

### UVB+Antibacterial (AB)

In one embodiment, said second light source is an antibacterial (AB) light. In one embodiment, said AB light is white light with a local peak wavelength of 405 nm. In one embodiment, said AB light and said UVB light synergistically suppresses bacteria. In other words, the bacteria suppressing benefits of UVB light can be combined certain wavelengths of violet light (e.g. 395, 405 nm), which are known to suppress bacteria, to provide white AB light.

### Half UVB+Full Spectrum

In one embodiment, said second light has a CRI of at least 80, or at least 85, or at least 90. In one embodiment, said emitted light has a CRI of at least 80, or at least 85, or at least 90. In one embodiment, said second light source is a Vigor Light system commercially available from Ecosense (see, also, PCT/US2019/043788).

### Controller

The controller functions to power the first and second light independently such that the contribution of each to the overall emitted light can be varied to optimize the health benefits of the lighting system.

### Dose

In one embodiment, the controller is configured to ensure that the first light delivers an appropriate dose of UVB. As discussed above, it is important to limit the dose of UVB directed at target. In one embodiment, the dose of said UVB directed at least one target 104 should be sufficient to cause vitamin D synthesis in said target. Although this dose can vary according to people's body types, in particular skin color, generally speaking, the dose should be no greater than 0.35 SED of said UVB light in an eight hour period. In a more particular embodiment, the dose is no greater than 0.1 to 0.15 SED of said UVB in a 15 min period.

### Timing to Reduce Detrimental Effect

In another embodiment, the controller is configured to time the UVB light to minimize the harmful effects of UVB the light. More specifically, the harmful effects of UVB light appear to be minimized at biological midday. Accordingly, in one embodiment, the UVB light is delivered during biological mid-day, e.g. 5-9 hours after habitual wake time, or based on feedback from the user (e.g., a wearable sensor measures the user's body clock).

In one embodiment, the lighting system of the present invention is configured to reinforce/realign a person's circadian rhythm before delivering the UVB dose. Accordingly, in one embodiment, said controller is configured to power said first light source to emit said UVB during high circadian stimulation of said target. In one embodiment, said high circadian stimulation of said target is based on time of day. In one embodiment, said second light source has at least a first mode in which said second light is a high EML light to stimulate circadian response, and wherein said dose is delivered when said second light source is in said first mode. In one embodiment, said high circadian stimulation of said target is based on (bio) feedback from target.

In one embodiment, said second light source has at least a first mode in which said second light is a high EML light to stimulate circadian response, and said controller is configured emit said second light in said first mode to align said target's circadian rhythm with a desired circadian rhythm, and to deliver said dose only after establishing said desired circadian rhythm.

### UVB+Wearables

In one embodiment, the dose of UVB is based on feedback from one or more users. More specifically, in one embodiment, at least one user is wearing a device that measures UVB exposure. In such an embodiment, said controller is responsive to at least one feedback signal of wearable of at least one occupant of space subjected to said emitted light, wherein said feedback signal is a measure of the UVB to which said at least one occupant has received over a given period of time. In one embodiment, there are multiple targets, each providing a feedback signal of individual UVB exposures. In one embodiment, said controller adjusts the output of said first light source such that no one of said multiple targets receives more than said 0.35 SED in an 8 hour period. In one embodiment, said controller signals a target who will not receive a sufficient does of UVB to spend time outside or spend time with an individual therapy lamp

In one embodiment, said wearable determines dosage based on pigment of skin. In another embodiment, an algorithm is used to determine skin color from an image of user skin tone to adjust the dosage.

These and other advantages maybe realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1. A lighting system for emitting emitted light, said light system comprising:
a first light source for emitting UVB light;
a second light source for emitting a second light, wherein said emitted light comprises a combination of said second light and said UVB light; and
a controller for said first light source configured to deliver a dose of said UVB to at least one target sufficient to cause vitamin D synthesis in said target.

2. The lighting system of claim 1, wherein said dose is no greater than 0.35 SED of said UVB light in an eight hour period.

3. The lighting system of claim 1, wherein said dose is no greater than 0.1 to 0.15 SED of said UVB in a 15 min period.

4. The lighting system of claim 1, wherein said second light comprising long red or NIR at a sufficient intensity at said target to be absorbed by cytochrome c oxidase enzymes.

5. The lighting system of claim 4, wherein said controller is configured to emit said second light before said dose is delivered to said target.

6. The lighting system of claim 4, wherein said controller causes said second light to be emitted to saturate said cytochrome c oxidase enzymes of said target prior to delivering said dose of UVB.

7. The lighting system of claim 4, wherein said controller is configured to emit said second light after said dose is delivered to said target.

8. The lighting system of claim 4, further comprising a booth for enclosing target and providing privacy, thereby allowing more skin area of the target to be exposed for receiving said dose.

9. The lighting system of claim 1, wherein said second light configured to alter said target's circadian rhythm to treat Seasonal affective disorder (SAD)

10. The lighting system of claim 9, wherein said second light source has at least two modes, a first mode in which said second light is a high EML light to stimulate circadian response, and a second mode in which said second light is a low EML light to minimize a circadian response.

11. The lighting system of claim 10, wherein said dose is delivered when said second light source is in said first mode.

12. The lighting system of claim 1, wherein said controller is configured to power said first light source to emit said UVB during high circadian stimulation of said target.

13. The lighting system of claim 12, wherein said high circadian stimulation of said target is based on time of day.

14. The lighting system of claim 12, wherein said second light source has at least a first mode in which said second light is a high EML light to stimulate circadian response, and wherein said dose is delivered when said second light source is in said first mode.

15. The lighting system of claim 12, wherein said high circadian stimulation of said target is based on feedback from target.

16. The lighting system of claim 15, wherein said second light source has at least a first mode in which said second light is a high EML light to stimulate circadian response, and said controller is configured emit said second light in said first mode to align said target's circadian rhythm with a desired circadian rhythm, and to deliver said dose only after establishing said desired circadian rhythm.

17. The lighting system of claim 1, wherein said controller is responsive to at least one feedback signal of wearable of at least one occupant of space subjected to said emitted light, wherein said feedback signal is a measure of the UVB to which said at least one occupant has received over a given period of time.

18. The lighting system of claim 17, wherein said wearable determines dosage based on pigment of skin.

19. The lighting system of claim 1, wherein said dose is based on skin color.

20. The lighting system of claim 19, wherein algorithm could be implemented to use camera image of user skin tone to adjust the dosage

21. The lighting system of claim 1, wherein said at least one target comprises multiple targets and wherein feedback signals include individual UVB exposures.

22. The lighting system of claim 21, wherein said controller adjusts the output of said first light source such that no one of said plurality of targets receives more than said 0.35 SED in an 8 hour period.

23. The lighting system of claim 22, wherein said controller signals a target who will not receive a sufficient does of UVB to spend time outside or spend time with an individual therapy lamp

24. The lighting system of claim 1, wherein said second light source is an antibacterial (AB) light

25. The lighting system of claim 24, wherein said AB light is white light with a local peak wavelength of 405 nm.

26. The lighting system of claim 24, wherein said AB light and said UVB light synergistically suppresses bacteria.

27. The lighting system of claim 1, wherein said second light has a CRI of at least 80.

28. The lighting system of claim 27, wherein said second light source is a Vigor

29. The lighting system of claim 1, wherein said UVB light has a peak wavelength of 290-315 nm.

30. The lighting system of claim 29, wherein said UVB light has a peak wavelength of 300 nm.
